(19) United States
(12) Patent Application Publication
(10) Pub. No.: US 2007/0129359 A1
(43) Pub. Date: Jun. 7, 2007

(54) Tetrahydronaphthalene derivatives, processes for their preparation and their use as antiinflammatory agents

(76) Inventors: Christoph Huwe, Berlin (DE); Werner Skuballa, Berlin (DE); Duy Nguyen, Berlin (DE); Heike Schaecke, Berlin (DE); Wheeseong Lee, Orinda, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/414,556

(22) Filed: May 1, 2006

Related U.S. Application Data
(63) Continuation of application No. PCT/EP06/03781, filed on Apr. 13, 2006.
(60) Provisional application No. 60/671,110, filed on Apr. 14, 2005.

(30) Foreign Application Priority Data
Apr. 14, 2005 (DE) 102005017326.8

(51) Int. Cl. A61K 31/538; A61K 31/498; A61K 31/4745; A61K 31/4704; A61K 31/404; A61K 31/4035

(52) U.S. Cl. 514/230.5; 514/248; 514/303; 514/309; 514/312; 514/367; 514/375; 514/406; 514/416; 514/417; 514/249; 514/300; 514/394; 544/105; 544/235; 544/353; 546/119; 546/122; 546/145; 546/153

(57) ABSTRACT

The invention relates to multiply substituted tetrahydronaphthalene derivatives of the formula (I) 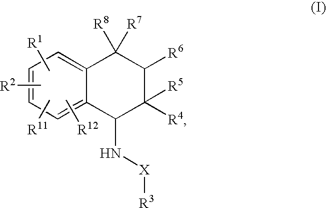 processes for their preparation and their use as anti-inflammatory agents.

This application is a continuation of PCT Application No. ______ filed Apr. 13, 2006 and also claims the benefit of the filing date of U.S. Provisional Application Ser. No. 60/671,110 filed Apr. 14, 2005, both of which are incorporated by reference herein.

### INTRODUCTION

The invention relates to tetrahydronaphthalene derivatives, processes for their preparation and their use as antiinflammatory agents.

Open-chain non-steroidal antiinflammatory agents are known in the art (DE 100 38 639 and WO 02/10143). These compounds show experimentally dissociations between antiinflammatory and unwanted metabolic effects and are superior to non-steroidal glucocorticoids described to date or exhibit at least as good an effect.

The present invention provides further non-steroidal antiinflammatory agents.

### BRIEF DESCRIPTION OF THE INVENTION

The present invention relates to compounds of the general formula (I) 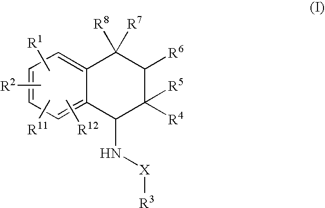
in which
R¹ and R² are independently of one another a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R⁹a group,
or R¹ and R² together form a group selected from the groups —O—(CH₂)—O—, —O—(CH₂)ₙ—CH₂—, —O—CH═CH—, —(CH₂)ₙ₊₂—, —NH—(CH₂)ₙ₊₁—, —N (C₁-C₃-alkyl)—(CH₂)ₙ₊₁— and —NH—N═CH—,
where n is 1 or 2, and the terminal oxygen atoms and/or carbon atoms and/or nitrogen atoms are linked to directly adjacent ring carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a C₁-C₁₀-alkyl group which is optionally substituted by 1 to 3 hydroxy groups, 1 to 3 halogen atoms, and/or 1 to 3 (C₁-C₁₀)-alkoxy groups, an optionally substituted (C₃-C₇)-cycloalkyl group, an optionally substituted heterocyclyl group, an optionally substituted aryl group, or a mono- or bicyclic heteroaryl group which is optionally substituted by one or more groups which are selected independently of one another from
   (C₁-C₅)-alkyl groups which themselves may optionally be substituted by 1 to 3 hydroxy or 1 to 3 —COOR¹³ groups,
   (C₁-C₅) -alkoxy groups,
   halogen atoms, hydroxy groups, —NR⁹R^{9a} groups,
and
   exo methylene groups and optionally comprises 1 to 4 nitrogen atoms and/or 1 to 2 oxygen atoms and/or 1 to 2 sulfur atoms and/or 1 to 2 keto groups, this group being linked by any position to the group X and possibly being optionally hydrogenated at one or more positions,
R⁴ is a hydroxy group, an —OR¹⁰ group or an —O(CO)R¹⁰ group,
R⁵ is a (C₁-C₁₀)-alkyl group which is optionally partly or completely fluorinated, a (C₃-C₇) cycloalkyl group, a (C₁-C₈)alkyl-(C₃-C₇) cycloalkyl group, a (C₂-C₈) alkenyl-(C₃-C₇) cycloalkyl group, a heterocyclyl group, a (C₃-C₈)alkylheterocyclyl group$ a (C₂-C₈)-alkenylheterocyclyl group, an aryl group, a (C₁-C₈) alkylaryl group, a (C₂-C₈) alkenylaryl group, a (C₂-C₈)-alkynylaryl group,
a mono- or bicyclic heteroaryl group which is optionally substituted by 1 to 2 keto groups, 1 to 2 (C₁-C₅)-alkyl groups, 1 to 2 (C₁-C₅)-alkoxy groups, 1 to 3 halogen atoms, and/or 1 to 2 exo methylene groups and comprises 1 to 3 nitrogen atoms and/or 1 to 2 oxygen atoms and/or 1 to 2 sulfur atoms,
a (C₁-C₈)alkylheteroaryl group, a (C₂-C₈)alkenylheteroaryl group, or a (C₂-C₈) alkynylheteroaryl group, this group being linked by any position to the tetrahydronaphthalene system and possibly being optionally hydrogenated at one or more positions,
R⁶ is a hydrogen atom, a halogen atom, or an optionally substituted (C₁-C₁₀)alkyl group,
R⁷ and R⁸ are independently of one another a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (c₃-C₆)-cyclalkyl ring; or
R⁶ and R⁷ together form a fused five- to eight-membered saturated or unsaturated carbocycle or heterocycle which is optionally substituted by 1 to 2 keto groups, 1 to 2 (C₁-C₅)-alkyl groups, 1 to 2 (C₁-C₅) -alkoxy groups, and/or 1 to 4 halogen atoms; or
R¹ and R⁸ together form a fused five- to eight-membered saturated or unsaturated carbocycle or heterocycle which is optionally substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a} are independently of one another a hydrogen atom, (C₁-C₁₀)-alkyl or —(CO)—(C₁-C₅)-alkyl,
R¹⁰ is a (C₁-C₁₀)-alkyl group or any hydroxy protective group,
R¹³ is a hydrogen atom or a (C₁-C₅)-alkyl group, and
X is a group —C(═O)—, —C(═S)—, —C(═O)—NH—, —C(═S)—NH—, —S(O)ₘ (where m=1 or 2), —C(═O)—O—, —C(═S)—O— or a group —(CH₂)ₚ (where p=1, 2 or 3), where if X comprises a carbonyl or thiocarbonyl function, this function is linked to the group —NH— in the general formula (I),
with the proviso that if R³ is an optionally substituted (C₁-C₁₀)-alkyl group, X cannot be a —(CH₂)ₚ-group;
   in the form of any stereoisomer or of a mixture of stereoisomers; or as pharmacologically acceptable salt or derivative.

The present invention further relates to processes for preparing compounds of the general formula (I) as described herein.

The present invention further relates to pharmaceutical compositions which include one or more compounds of the general formula (I) in combination with one or more pharmaceutical carriers or excipients.

The present invention additionally relates to the use of the compounds of the general formula (I) for manufacturing pharmaceutical compositions having an antiinflammatory effect.

The present invention further relates to compounds of the general formula (IV) 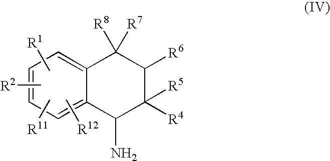
in which the substituents R¹ to R² have the abovementioned meanings, and to the use of these compounds for preparing compounds of the general formula (I) as described above.

### DETAILED DESCRIPTION OF THE INVENTION

Definitions

The term halogen atom or halogen means a fluorine, chlorine, bromine or iodine atom. A fluorine, chlorine or bromine atom is preferred.

The alkyl groups mentioned in the definitions of the general formula (I) may be straight-chain or branched and are for example a methyl, ethyl, n-propyl, isopropyl, n-butyl, isobutyl, tert-butyl or n-pentyl, 2,2-dimethylpropyl, 2-methylbutyl or 3-methylbutyl group, and the hexyl, heptyl, nonyl, decyl group and derivatives thereof branched in any way. Alkyl groups which comprise 1 to 10, 1 to 8 or 1 to 5 carbon atoms are preferred. A methyl or ethyl group is particularly preferred.

The abovementioned alkyl groups may optionally be substituted by 1 to 5, preferably 1 to 3, groups which are selected independently of one another from hydroxy, cyano, nitro, —COOR¹³, (C₁-C₅)-alkoxy groups, halogen atoms, —NR⁹R^{9a}, a partly or completely fluorinated (C₁-C₃)-alkyl group. The alkyl groups may preferably be substituted by 1 to 3 halogen atoms and/or 1 to 3 hydroxy and/or 1 to 3 cyano and/or 1 to 3 —COOR¹³ groups. Fluorine atom, hydroxy, methoxy and/or cyano groups represent a particularly preferred subgroup of substituents.

1 to 3 hydroxy and/or 1 to 3 —COOR¹³ groups are a further particularly preferred group of substituents for the alkyl groups. Hydroxy groups are particularly preferred in this connection.

Examples of a suitable partly or completely fluorinated alkyl group are the following partly or completely fluorinated following groups: fluoromethyl, difluoromethyl, trifluoromethyl, fluoroethyl, 1,1-difluoroethyl, 1,2-difluoroethyl, 1,1,1-trifluoroethyl, tetrafluoroethyl, pentafluoroethyl. Of these, the trifluoromethyl or the pentafluoroethyl are preferred. The completely fluorinated group is also called perfluoroalkyl group. The reagents which are optionally employed during the synthesis can be purchased, or the published syntheses of the corresponding reagents belong to the prior art, or published syntheses can be applied analogously.

The alkenyl groups have at least one C═C double bond and may be straight-chain or branched. Alkenyl groups having 2 to 8 carbon atoms are preferred.

The alkynyl groups have at least one C═C triple bond and may be straight-chain or branched. Alkynyl groups having 2 to 8 carbon atoms are preferred.

The alkoxy groups mentioned in the definitions of the general formula (I) may be straight-chain or branched and be for example a methoxy, ethoxy, n-propoxy, isopropoxy, n-butoxy, isobutoxy, tert-butoxy or n-pentoxy, 2,2-dimethylpropoxy, 2-methylbutoxy or 3-methylbutoxy group. C₁-C₅- and C₁-C₃-, C₁-C₈-, and C₁-C₁₀-alkoxy groups are preferred. A methoxy or ethoxy group is particularly preferred.

The alkylthio groups mentioned in the definitions of the general formula (I) may be straight-chain or branched and are for example a methylthio, ethylthio, n-propylthio, isopropylthio, n-butylthio, isobutylthio, tert-butylthio or n-pentylthio, 2,2-dimethylpropylthio, 2-methylbutylthio or 3-methylbutylthio group. C₁-C₅-Alkylthio groups are preferred. A methylthio or ethylthio group is particularly preferred.

The alkoxy and alkylthio groups described above may have on their alkyl groups the same substituents which have been described hereinbefore for the alkyl groups in general. Preferred substituents for alkoxy and alkylthio groups are selected independently of one another from halogen atoms (especially fluorine and/or chlorine), hydroxy and cyano groups.

The substituent —NR⁹R^{9a} means for example —NH₂, —NH(CH₃), —N(CH₃)₂, —NH(C₂H₅)₂, —N (C₂H₇), —NH(C₃H₇)₂, —N(C₃-H₇)₂, —NH(C₄-H₉), —N(C₄H₉)₂, —NH(C₅-H₁₁), —N(C₅H₁₁)₂, —NH(CO)CH₃, NH(CO)C₂H₅, —NH(CO)C₃H₇, —NH(CO)C₄H₉, —NH(CO)C₅H₁₁.

The cycloalkyl group means a saturated cyclic group which is optionally substituted by one or more groups selected from hydroxy groups, halogen atoms, (C₁-C₅)-alkyl groups, (C₁-C₅)-alkoxy groups, —NR⁹R^{9a} groups, —COOR¹³ groups, —CHO, cyano and has 3 to 7 ring carbon atoms such as, for example, cyclopropyl, methyl-cyclopropyl, cyclobutyl, methylcyclobutyl, cyclopentyl, methylcyclopentyl, cyclohexyl, methylcyclohexyl, cycloheptyl, methylcycloheptyl.

A (C₁-C₈)alkyl-(C₃-C₇) cycloalkyl group R⁵ means a cycloalkyl group (as defined above) which is linked via a straight-chain or branched (C₁-C₈)-alkyl unit (as defined above) to the ring system. Examples of such groups are —(CH₂)-cycloalkyl, —(C₂H₄) -cycloalkyl, —(C₃H₆)-cycloalkyl, —(C₄H₈)-cycloalkyl, —(C₅H₁₀-cyclo-alkyl, where cycloalkyl is defined as described above.

A (C₂-C₈)alkenyl-(C₃-C₇)cycloalkyl group R⁵ means a cycloalkyl group (as defined above) which is linked via a straight-chain or branched (C₂-C₈)-alkenyl unit to the ring system. Examples of such groups are —(CH═CH)-cycloalkyl, —[C(CH₃)═CH]-cycloalkyl, —[CH═C(CH₃)]-cycloalkyl, —(CH═CH—CH₂)-cycloalkyl, —(CH₂—CH═CH)-cycloalkyl, —(CH═CH—CH₂—CH₂)-cycloalkyl, —(CH₂—CH═CH—CH₂)-cycloalkyl, —(CH₂—CH₂—CH═CH)-cycloalkyl, —(C(CH₃)═CH—CH₂)-cycloalkyl, —(CH═C(CH₃)—CH₂)-cyclo-alkyl.

An alkylidene or exo alkylidene group means a group having 1 to 10 carbon atoms which is bonded via an exo double bond to the system (ring or chain). (C₁-C₅)- and (C₁-C₃)-alkylidene is preferred, and exo methylene is particularly preferred.

The heterocyclyl group is a cyclic, non-aromatic group comprising one or more heteroatoms and may be for example pyrrolidine, imidazolidine, pyrazolidine, piperidine. Perhydroquinoline and perhydroisoquinoline are also included in the heterocyclyl groups of the invention.

Examples of suitable substituents for heterocyclyl and heteroaryl groups are substituents from the following group: optionally substituted C₁-C₅-alkyl groups, hydroxy, (C₁-C₅)-alkoxy, —NR⁹R^{9a}, halogen, cyano, —COOR¹³, —CHO. The substituents may optionally also be bonded to the nitrogen atom of the heterocyclyl or heteroaryl group; N-oxides are also included in the definition.

Aryl groups in the context of the invention are aromatic or partly aromatic carbocyclic groups having 6 to 14 carbon atoms which have one ring, such as, for example, phenyl or phenylene, or a plurality of fused rings, such as, for example, naphthyl or anthranyl. Examples which may be mentioned are phenyl, naphthyl, tetralinyl, anthranyl, indanyl, and indenyl. The optionally substituted phenyl group and the naphthyl group are preferred.

The aryl groups may be substituted at any suitable position leading to a stable compound by one or more radicals from the group of hydroxy, halogen, C₁-C₅-alkyl which is optionally substituted by 1 to 3 hydroxy groups or —COOR³ groups, or C₁-c₅-alkoxy, cyano, —CF₃ and nitro.

The aryl groups may be partly hydrogenated and then, in addition or as alternative to the substituents detailed above, also carry keto and/or exo alkylidene Partly hydrogenated phenyl means for example cyclohexadienyl, cyclohexenyl, cyclohexyl. A partly hydrogenated substituted naphthalene system is for example 1-tetralone or 2-tetralone.

A (C₁-C₈)alkylaryl group is an aryl group as already described above which is linked via a straight-chain or branched (C₁-C₈)-alkyl unit (as defined above) to the ring system.

A (C₂-C₈)alkenylaryl group is an aryl group as already described above which is linked via a straight-chain or branched (C₂-C₈)-alkenyl unit (as defined above) to the ring system.

A (C₂-C₈)alkynylaryl group is an aryl group as already described above which is linked via a straight-chain or branched (C₂-C₈)-alkynyl unit (as defined above) to the ring system.

The mono- or bicyclic heteroaryl group may optionally comprise 1 to 9 groups selected from nitrogen atoms, oxygen atoms, sulfur atoms or keto groups, of which a maximum of 4 nitrogen atoms, a maximum of 2 oxygen atoms, a maximum of 2 sulfur atoms and/or a maximum of 2 keto groups may be present. Every subcombination of these groups is possible. The heteroaryl group may be hydrogenated at one or more positions.

Monocyclic heteroaryl groups may be for example pyridine, pyrazine, pyrimidine, pyridazine, triazine, azaindolizine, 2H- and 4H-pyran, 2H- and 4H-thiopyran, furan, thiophene, 1H- and 4H-pyrazole, 1H- and 2H-pyrrole, oxazole, thiazole, furazan, 1H- and 4H-imidazole, isoxazole, isothiazole, oxadiazole, triazole, tetrazole, thiadiazole.

Bicyclic heteroaryl groups may be for example phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, benzo[b]thienyl, benzo[c]thienyl, dihydroisoquinolinyl, dihydroquinolinyl, benzoxazinonyl, phthalazinonyl, dihydrophthalazinonyl, quinolinyl, isoquinolinyl, quinolonyl, isoquinolonyl, quinazolinyl, quinoxalinyl, cinnolinyl, phthalazinyl, dihydrophthalazinyl, 1,7- or 1,8-naphthyridinyl, coumarinyl, isocoumarinyl, indolizinyl, isobenzofuranyl, azaindolyl, azaisoindolyl, pyrazolo[1,5-a]pyridinyl, furanopyridyl, furanopyrimidinyl, furanopyrazinyl, furanopyridazinyl, dihydrobenzofuranyl, dihydrofuranopyridyl, dihydrofuranopyrimidinyl, dihydrofuranopyrazinyl, dihydrofuranopyridazinyl, dihydrobenzofuranyl group.

If the heteroaryl groups are partly or completely hydrogenated, the present invention includes compounds of the general formula (I) in which R³ is for example tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylideneaminyl, chromanyl, isochromanyl, thiochromanyl, decahydroquinolinyl, tetrahydroquinolinyl, dihydro-quinclinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz-[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinozolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5)naphthyridyl, 1H-[1,5]naphthyrid-4-onyl , 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, 2,2-dihydro-1H-pyrrolo[2,3-b]pyridin-3-onyl.

The mono- or bicyclic heteroaryl group may optionally be substituted by one or more substituents selected from C₁-C₅-alkyl groups which are optionally substituted by 1 to 3 hydroxy groups or 1 to 3 —COOR¹³ groups, or C₁-C₅-alkoxy groups, halogen atoms, and/or exo methylene groups. The substituents may, if possible, optionally also be bonded directly to the heteroatom (e.g. to the nitrogen atom) The present invention also includes N-oxides.

A (C₁-C₈)alkylheteroaryl group is a heteroaryl group as already described above which is linked via a straight-chain or branched (C₁-CO) -alkyl unit (as defined above) to the ring system.

A (C₂-C₈)alkenylheteroaryl group is a heteroaryl group as already described above which is linked via a straight-chain or branched (C₂-C₈)-alkenyl unit (as defined above) to the ring system.

A (C₂-C₈)alkynylheteroaryl group is a heteroaryl group as already described above which is linked via a straight-chain or branched (C₂-C₈)-alkynyl unit (as defined above) to the ring system.

A (C₂-C₈)alkylheterocyclyl group is a heterocyclyl group as already described above which is linked via a straight-chain or branched (C₁-C₈)-alkyl unit (as defined above) to the ring system.

A (C₂-Ca)alkenylheterocyclyl group is a heterocyclyl group as already described above which is linked via a straight-chain or branched (C₂-C₈)-alkenyl unit (as defined above) to the ring system.

Suitable hydroxy protective groups are all conventional hydroxy protective groups known to the skilled worker, in particular silyl ethers or esters of organic C₁-C₁₀ acids, C₁-C₅ ethers, benzyl ethers or benzyl esters. Conventional hydroxy protective groups are described in detail in T. W. Greene, P. G. M. Wuts “Protective Groups in Organic Synthesis”, 2nd edition, John Wiley & Sons, 1991). The protective groups are preferably alkyl-, aryl- or mixed alkylaryl-substituted silyl groups, e.g. the trimethylsilyl (TMS), triethylsilyl (TES), tert-butyldimethylsilyl (TBDMS), tert-butyldiphenylsilyl (TBDPS) or triisopropylsilyl groups (TIPS) or other customary hydroxy protective groups (e.g. methoxymethyl, methoxyethoxymethyl, ethoxyethyl, tetrahydrofuranyl, tetrahydropyranyl groups).

The compounds of the invention of the general formula (I) may, owing to the presence of centers of asymmetry, exist as stereoisomers. The present invention relates to all possible diastereoisomers both as racemates and in enantiopure form. The term stereoisomers also includes all possible diastereoisomers and regioisomers and tautomers (e.g. keto-enol tautomers) in which the stereoisomers of the invention may exist, and to which the invention therefore likewise relates.

The compounds of the invention may also be in the form of salts with pharmacologically acceptable anions, for example in the form of the hydrochloride, sulfate, nitrate, phosphate, pivalate, maleate, fumarate, tartrate, benzoate, mesylate, citrate or succinate.

The invention also includes pharmacologically suitable derivatives or prodrugs of the compounds of the general formula (I). Derivatives or prodrugs refers for example to esters, ethers or amides of the compounds of the general formula (I) or other compounds which metabolize in the body to compounds of the general formula (I). Suitable compounds are listed for example in Hans Bundgaard (Editor), Design of Prodrugs, Elsevier, Amsterdam 1985.

Preferred Embodiments

A subgroup of compounds of the invention of the general formula (I) are those compounds in which R⁷ and R⁸ are independently of one another a hydrogen atom, a halogen atom, an optionally substituted (C₁-C₁₀)-alkyl group, a cyano group, together a (C₁-C₁₀)-alkylidene group or together with the carbon atom of the tetrahydro-naphthalene system an optionally substituted (C₃-C₆)-cycloalkyl ring.

A further subgroup of compounds of the invention of the general formula (I) are those compounds in which R⁶ and R⁷ together form a fused five- to eight-membered saturated or unsaturated carbocycle or heterocycle which is optionally substituted by 1 to 2 keto groups, 1 to 2 (C₁-C₅)-alkyl groups, 1 to 2 (C₁-C₅)-alkoxy groups, and/or I to 4 halogen atoms.

A further subgroup of compounds of the invention of the general formula (I) are those compounds in which R¹ and R⁸ together form a fused five- to eight-membered saturated or unsaturated carbocycle or heterocycle which is optionally substituted by 1 to 2 keto groups, 1 to 2 (C₁-C₅)-alkyl groups, 1 to 2 (C₁-C₅)-alkoxy groups, and/or 1 to 4 halogen atoms.

A further subgroup of compounds of the invention of the general formula (I) are those compounds in which R¹ and R² are independently of one another a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group.

A further subgroup of compounds of the invention of the general formula (I) are those compounds in which or R¹ and R² together form a group selected from the groups —O—(CH₂)ₙ—O, —O—(CH₂)ₙ—CH₂—, —O—CH═CH—, —(CH₂)ₙ₊₂—, —NH—(CH₂)ₙ₊₁—, —N(C₁-C₃-alkyl)—(CH₂)ₙ₊₁— and —NH—N═CH—, where n=1 or 2, and the terminal oxygen atoms and/or carbon atoms and/or nitrogen atoms are linked to directly adjacent ring carbon atoms.

A preferred group of compounds of the general formula (I) are those compounds in which X is a group —C(═O)—, —C(═O)—NH—, —SO₂— or —CH₂—.

A further preferred group of compounds of the general 35 formula (I) are those compounds in which R⁴ is a hydroxy group or a group —OR¹⁰. Those compounds in which R⁴ is a hydroxy group are particularly preferred.

A further preferred group of compounds of the general formula (I) are those compounds in which R⁵ is a (C₁-C₅)-alkyl group or a partly or completely fluorinated (C₁-C₅)-alkyl group, an aryl group, a (C₁-C₈)alkylaryl group, a (C₂-C₈)alkenylaryl group, a (C₃-C₇) cycloalkyl group, a (C₁-C₈)alkyl (C₃-C7) cycloalkyl group or a (C₂-C₈)alkenyl (C₃-C₇) cycloalkyl group. Compounds which are preferred in this connection are those in which R⁵ is a (C₁-C₅)-alkyl group or a partly or completely fluorinated (C₁-C₅)-alkyl group. Particularly preferred compounds in this connection are those in which R⁵ is a trifluoromethyl or a pentafluoroethyl group.

A further preferred group of compounds of the general formula (I) are those compounds in which R⁷ is a halogen atom or an optionally substituted methyl or ethyl group.

A further preferred group of compounds of the general formula (I) are those compounds in which R⁷ and R⁸ are each a methyl group, or together with the carbon atom of the tetrahydronaphthalene system form a cyclopropyl group. Particularly preferred compounds are those in which R⁷ and R⁸ are each a methyl group.

A further preferred group of compounds of the general formula (I) are those compounds in which R³ is an optionally substituted aryl or heteroaryl group. Particularly preferred compounds in this connection are those in which the aryl or heteroaryl group is selected from the group consisting of naphthyl, benzofuranyl, pyrazolol[1,5-a]pyridinyl, phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, chromanyl, isochromanyl, quinazolinyl, quinoxalinyl, cinnolinyl, phthalazinyl, 1,7- or 1,8-naphthyridinyl, dihydroindolonyl, dihydroisoindolonyl, benzimidazole or indolyl. Particularly preferred groups in this connection are naphthyl, benzofuranyl, quinoxalinyl, and pyrazolo[1,5-a]pyridinyl.

A subgroup of compounds of the general formula (I) are those compounds in which the substituents R¹¹ nd R¹² are each independently of one another a hydrogen atom, a halogen atom, in particular fluorine, a cyano or a methoxy group.

In a preferred subgroup, the substituents R¹¹ and R¹² are each a hydrogen atom.

A particularly preferred group of compounds of the general formula (I) as defined above are those compounds in which R¹, R², R¹¹, and R¹² are independently of one another a hydrogen atom, a halogen atom, a hydroxy group, an optionally substituted (C₁-C₁₀)-alkyl group, or a (C₁-C₁₀)-alkoxy group, R³ is an optionally substituted aryl or heteroaryl group, R⁴ is a hydroxy group, an —OR¹⁰ group or an —O(CO)R¹⁰ group, R⁵ is a (C₁-C₁₀)alkyl group which is optionally partly or completely fluorinated, R⁶ is a hydrogen atom, a halogen atom or an optionally substituted (C₁-C₁₀)-alkyl group, R⁷ and R⁸ are independently of one another an optionally substituted (C₁-C₁₀)-alkyl group, together a (C₁-C₁₀)-alkylidene group or together with the carbon atom of the tetrahydronaphthalene system an optionally substituted (C₃-C₆)-cycloalkyl ring, R¹⁰ is a (C₁-C₁₀)-alkyl group, and X is the group —C(═O)—, —C(═O)—NH—, —S(O)ₘ— (where m equals 1 or 2), or —(CH₂)ₚ— (where p equals 1, 2 or 3).

A very particularly preferred group of compounds of the general formula (I) as defined above are those compounds in which R¹ and R² are each independently of one another a hydrogen atom, a hydroxy group or a methoxy group, R¹¹ and R¹² are each a hydrogen atom, R³ is a naphthyl, benzofuranyl, quinoxalinyl or pyrazolo[1,5-a]pyridinyl group, R⁴ is a hydroxy group, R⁵ is a trifluoromethyl group, R⁶ is a hydrogen atom, R⁷ and R⁸ are each a methyl group, and X is one of the groups —C(═O)—, —C(═O)—NH—, —SO₂— and —CH₂—.

Every further possible combination of the abovementioned subgroups and of substituents indicated as preferred with their general and/or specific meanings is likewise to be regarded as encompassed by the present invention.

Preparation Processes

The compounds of the invention of the general formula (I) can be obtained in various ways. The preparation processes described below likewise form a part of the present invention.

Unless indicated otherwise, the substituents used in the process descriptions below have the same meaning as above in the section “Brief description of the invention”, including the definitions stated in the section “Detailed description of the invention”.

One process according to the invention (process A) for preparing compounds of the general formula (I) is characterized in that
a) an open-chain carbonyl compound of the general formula (II) 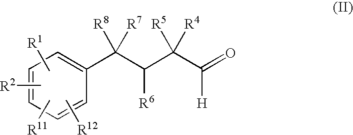
is cyclized where appropriate with addition of an inorganic or organic acid or Lewis acid to a compound of the general formula (III) 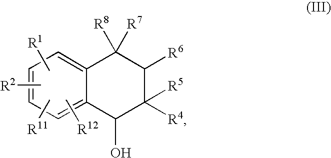
b) the compound of the general formula (III) is converted by processes known in the art for replacing the hydroxy group by the amino group into a compound of the general formula (IV) 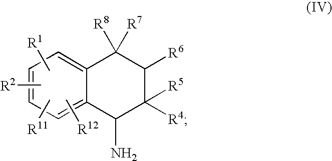
and
c) the compound of the general formula (IV) is converted by reaction with a compound selected from compounds of the general formulae R³-X-Nu (where Nu is a nucleofugic group), R³—N—C—O or R³—N═C═S, and where appropriate subsequent replacement of a carbonyl oxygen atom by sulfur by processes known in the art, into a compound of the general formula (I).

The substituents R¹ to R¹² have the meanings indicated above.

Replacement of the hydroxy group by the amino group in step b) indicated above can be achieved for example by converting the compound of the general formula (III) by processes known in the art (nucleophilic substitution) into the corresponding azide, which can be reduced under suitable conditions to the primary amine of the general formula (IV).

A further possibility for introducing the amino group consists of reacting the compound of the general formula (III) with Burgess' reagent (Tetrahedron Lett. 2002, 43, 3887-3890) and subsequent cleavage of the resulting heterocycle, which is obtainable by processes known in the art.

The optional replacement of a carbonyl oxygen atom by sulfur which is described in step c) is known in the art and can be achieved for example by reaction with Lawesson's reagent or phosphorus pentasulfide.

Examples of suitable nucleofugic groups Nu in the compound R³-X-Nu used in step c) are halogen atoms or leaving groups such as, for example, the acetate, tosylate, mesylate or triflate group. The compounds R³-X-Nu thus belong for example to the classes of the halides of carboxylic acids, sulfonic acids, or sulfinic acids, or of mixed anhydrides of these acids, and to the esters of chloroformic acid, of toluenesulfonic acid, of methylsulfonic acid and of trifluoromethylsulfonic acid.

A further process according to the invention (process B) for preparing compounds of the general formula (I) consists of reacting a compound of the general formula (IV) as described above with a compound of the general formula R³—CHO, and reducing the resulting imine.

Finally, a further process according to the invention (process C) for preparing compounds of the general formula (I) consists of reacting a compound of the general formula (IV) as described above with phosgene or thiophobgene, and subsequently reacting the resulting isocyanate or isothiocyanate with compounds of the general formula R³—OH or R³—NH₂ to give a compound of the general formula (I), where X has the meaning —C(═O)—NH—, —C(—S)—NH— or —C(═O)—O— indicated in claim 1.

A further process according to the invention (process D) for preparing a compound of the general formula (I) is characterized in that
a) a compound of the general formula (VI) 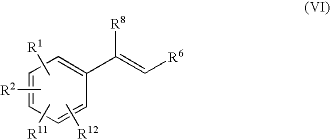
   where R⁸ is a (C₁-C₁₀)-alkyl group, is reacted with an α-keto carboxylic acid or an α-keto carboxylic ester of the general formula R⁵—C(═O)—COOR¹³ in an ene reaction in the presence of an optionally chiral Lewis acid to give a compound of the general formula (VII) 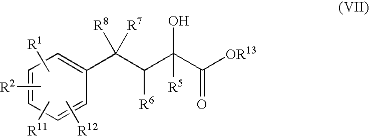
   in which R⁷ and R⁸ together have the meaning of a (C₁-C₁₀)-alkylidene group,
b) the compound of the general formula (VII) is reduced to an aldehyde of the general formula (IIa), 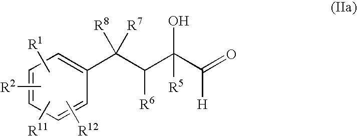
   in which R⁷ and R⁸ together have the meaning of a (C₁-C₁₀)-alkylidene group, and
c) the aldehyde (IIa) is converted in analogy to process A indicated above into a compound of the general formula (I) in which R⁷ and R⁸ together have the meaning of a (C₁-C₁₀)-alkylidene group.

The substituents R¹ to R¹³ have the meanings indicated above.

Processes for selective reduction of a carboxylic acid or of a carboxylic ester to the aldehyde, as used in step b) of process D, are known in the art.

It is optionally possible to include in process D further reaction steps to modify R, and Ra. Thus, for example, the (C₁-C₁₀)-alkylidene group in intermediate (IIa) can be hydrogenated, thus providing compounds of the general formula (I) in which one of the radicals R⁷ and R⁸ has the meaning of a hydrogen atom, whereas the other radical is a (C₁-C₁₀)-alkyl group.

The (C₁-C₁₀)-alkylidene group in intermediate (IIa) can also be employed as substrate for a hydrohalogenation. The final products of the synthesis resulting in this case are compounds of the general formula (I) in which one of the radicals R⁷ and R⁸ has the meaning of a halogen atom, or else the other radical is a (C₁-C₁₀)-alkyl group.

Finally, it is also possible to carry out cycloaddition reactions on the (C₁-C₁₀)-alkylidene group in intermediate (IIa). Particularly preferred in this connection are cyclopropanation reactions which afford as final products of the synthesis compounds of the general formula (I) in which the radicals R⁷ and R⁸ have, together with the ring carbon atom, the meaning of an (optionally substituted) cyclopropane ring.

It is directly evident to the skilled worker that these modifications to the radicals R⁷ and R⁸ in process D do not necessarily have to be carried out on intermediate (IIa) but can also where appropriate be carried out at a later time in the complete synthesis. These variations in the sequence of the reaction steps are likewise included in the present invention.

Biological Activity

The antiinflammatory effect of the compounds of the general formula (I) is tested in an animal experiment by testing in the croton oil-induced inflammation in the rat and mouse (J. Exp. Med. (1995), 182, 99-108). For this purpose, croton oil is applied in ethanolic solution topically to the ears of the animals. The test substances are likewise administered topically or systemically at the same time as or two hours before the croton oil. After 16-24 hours, the ear weight is measured as a measure of the inflammatory edema, the peroxidase activity is measured as a measure of the migration in of granulocytes and the elastase activity is measured as a measure of the migration in of neutrophilic granulocytes. In this test, the compounds of the general formula (I) inhibit the three abovementioned parameters of inflammation both after topical and after systemic administration.

The binding of the substances to the glucocorticoid receptor (GR) and further steroid hormone receptors (mineral corticoid receptor (MR), progesterone receptor (PR) and androgen receptor (AR)) is examined with the aid of recombinantly prepared receptors. Cytosol preparations of Sf9 cells which had been infected with recombinant baculoviruses which code for the GR are employed for the binding studies. Compared with the reference substance [³H]-dexamethasone, the substances show a high affinity for the GR. Thus, an IC₅₀(GR)=36 nM and IC₅₀(PR)>1 μM was measured for the compound of example 3L.

The essential molecular mechanism for the antiinflammatory effect of glucocorticoids is regarded as the GR-mediated inhibition of the transcription of cytokines, adhesion molecules, enzymes and other pro-inflammatory factors. This inhibition is brought about by an interaction of the GR with other transcription factors, e.g. AP-1 and NF-kappa-B (for review, see Cato ACB and Wade E, BioEssays 18, 371-378 1996).

The compounds of the invention of the general formula (I) inhibit the secretion, induced by lipopolysaccharide (LPS), of the cytokine IL-8 in the human THP-1 monocyte cell line. The concentration of the cytokines was determined in the supernatant using a commercially available ELISA kit. The compound of example 3E exhibited an IC₅₀(ILB)=1 μmol inhibition at an 11% efficiency with regard to [³H]-dexamethasone as standard.

One of the commonest unwanted effects of a glucocorticoid therapy is the so-called “steroid diabetes” [cf. Hatz, H J, Glucocorticoide: Immunologische Grundlagen, Pharmakologie und Therapierichtlinien, Wissenschaftliche Verlagsgesellachaft mbH, Stuttgart, 1998). The cause of this is stimulation of gluconeogenesis in the liver by induction of the enzymes responsible therefor and by free amino acids resulting from the breakdown of proteins (catabolic effect of the glucocorticoids). A key enzyme in catabolic metabolism in the liver is tyrosine aminotransferase (TAT). The activity of this enzyme can be determined by photometry on liver homogenates and represents a good measure of the unwanted metabolic effects of glucocorticoids. To measure the TAT induction, the animals are sacrificed 8 hours after administration of the test substances, the livers are removed, and the TAT activity in the homogenate is measured. In this test, the compounds of the general formula (I) induce, in doses in which they have antiinflammatory activity, tyrosine aminotransferase to only a small extent or not at all.

Medical Indications

Owing to their antiinflammatory and additional anti-allergic, immunosuppressive and antiproliferative effect, the compounds of the invention of the general formula (I) can be used as medicaments for the treatment or prophylaxis of the following pathological states in patients, especially mammals and preferably humans.

In this connection, the term “DISORDER” stands for the following indications:
(i) pulmonary disorders associated with inflammatory, allergic and/or proliferative processes:
   chronic obstructive lung disorders of any origin, especially bronchial asthma
   bronchitis of varying origin
   all types of restrictive lung disorders, especially allergic alveolitis,
   all types of pulmonary edema, especially toxic pulmonary edema
   sarcoidoses and granulomatoses, especially Boeck's disease
(ii) rheumatic disorders/autoimmune diseases/joint disorders associated with inflammatory, allergic and/or proliferative processes:
   all types of rheumatic disorders, especially rheumatoid arthritis, acute rheumatic fever, polymyalgia rheumatica
   reactive arthritis
   inflammatory soft tissue disorders of other origin
   arthritic symptoms associated with degenerative joint disorders (arthroses)
   traumatic arthritides
   collagenoses of any origin, e.g. systemic lupus erythematosus, scleroderma, polymyositis, dermatomyositis, Sjögren's syndrome, Still's syndrome, Felty's syndrome
(iii) allergies associated with inflammatory and/or proliferative processes:
   all types of allergic reactions, e.g. angioedema, hayfever, insect bite, allergic reactions to drugs, blood derivatives, contrast agents etc., anaphylactic shock, urticaria, contact dermatitis
(iv) vessel inflammations (vasculitides)
   polyarteritis nodosa, temporal arteritis, erythema nodosum
(v) dermatological disorders associated with inflammatory, allergic and/or proliferative processes:
   atopic dermatitis (especially in children)
   psoriasis
   pityriasis rubra pilaris
   erythematous disorders induced by various noxae, e.g. radiation, chemicals, burns, etc. bullous dermatoses
   allergic keratitis, uveitis, iritis,
   conjunctivitis
   blepharitis
   optic neuritis
   chorioditis
   sympathetic ophthalmia
(xi) ear-nose-throat disorders associated with inflammatory, allergic and/or proliferative processes:
   allergic rhinitis, hayfever
   otitis externa, e.g. caused by contact exema, infection etc.
   otitis media
(xii) neurological disorders associated with inflammatory, allergic and/or proliferative processes:
   cerebral edema, especially tumor-related cerebral edema
   multiple sclerosis
   acute encephalomyelitis
   meningitis
   various types of seizures, e.g. infantile spasms
(xiii) blood disorders associated with inflammatory, allergic and/or proliferative processes:
   acquired hemolytic anemia
   idiopathic thrombocytopenia
(xiv) neoplastic disorders associated with inflammatory, allergic and/or proliferative processes:
   acute lymphatic leukemia
   malignant lymphomas
   lymphogranulomatoses
   lymphosarcomas
   extensive metastases, especially associated with breast, bronchial and prostate carcinomas
   lichenoid disorders
   pruritus (e.g. of allergic origin)
   seborrheic eczema
   rosacea
   pemphigus vulgaris
   erythema multiforme exudativum
   balanitis
   vulvitis
   hair loss such as alopecia areata
   cutaneous T-cell lymphomas
(vi) renal disorders associated with inflammatory, allergic and/or proliferative processes:
   nephrotic syndrome
   all nephritides
(vii) liver disorders associated with inflammatory, allergic and/or proliferative processes;
   acute liver cell necrosis
   acute hepatitis of varying origin, e.g. viral, toxic; drug-induced
   chronic aggressive and/or chronic intermittent hepatitis
(viii) gastrointestinal disorders , associated with inflammatory, allergic and/or proliferative processes:
   regional enteritis (Crohn's disease)
   ulcerative colitis
   gastritis
   reflux esophagitis
   gastroenteritides of other origin, e.g. indigenous sprue
(ix) proctological disorders associated with inflammatory, allergic and/or proliferative processes:
   anal eczema
   fissures
   hemorrhoids
   idiopathic proctitis
(x) ocular disorders associated with inflammatory, allergic and/or proliferative processes:
(xv) endocrine disorders associated with inflammatory, allergic and/or proliferative processes
   endocrine orbitopathy
   thyrotoxic crisis
   de Quervain's thyroiditis
   Hashimoto's thyroiditis
   Basedow's disease
(xvi) organ and tissue transplantations, graft-versus-hose disease
(xvii) severe states of shock, e.g. anaphylactic shock, systemic inflammatory response syndrome (SIRS)
(xviii) emesis associated with inflammatory, allergic and/or proliferative processes:
   e.g. in combination with a 5-HT3 antagonist in cytostic-related vomiting.
(xix) pain of inflammatory origin, e.g. lumbago
(xx) replacement therapy for:
   congenital primary adrenal insufficiency, e.g. congenital adrenogenital syndrome
   acquired primary adrenal insufficiency, e.g. Addison's disease, autoimmune adrenalitis, post-infection, tumors, metastases etc.
   congenital secondary adrenal insufficiency, e.g. congenital hypopituitarism
   acquired secondary adrenal insufficiency, e.g. post-infection, tumors etc.
      Medicaments comprising stereoisomers of the general formula I show a particular efficacy for the following disorders:
1. lung disorders
2. rheumatic disorders/autoimmune diseases
3. dermatological disorders
4. degenerative joint disorders
5. vessel inflammations
6. graft versus host disease
1 7. severe states of shock
8. emesis associated with inflammatory, allergic and/or proliferative processes
9. inflammation-related pain.

In addition, the compounds of the invention of the general formula (I) can be employed for the therapy and prophylaxis of further pathological states which are not mentioned above but for which synthetic glucocorticoids are currently used (concerning this, see Hatz, H J, Glucocorticoide: Immunologische Grundlagen, Pharmakologie und Therapierichtlinien, Wissenschafliche Verlagsgesellachaft mbH, Stuttgart, 1998).

All the aforementioned indications are described in detail in Hatz, H J, Glucocorticoide: Immunologische Grundlagen, Pharmakologie und Therapierichtlinien, Wissenschafliche Verlagsgesellachaft mbH, Stuttgart, 1998).

The suitable dose for a therapeutic effect in the abovementioned pathological states varies and depends for example on the potency of the compound of the general formula (I), the patient (e.g. height, weight, gender, etc.), the mode of administration and the nature and severity of the conditions to be treated, and the use as prophylactic or therapeutic agent.

The invention relates to the use of the claimed compounds for manufacturing a pharmaceutical composition.

The invention further provides:
(i) the use of one of the compounds of the invention of the general formula (I) or mixture thereof for manufacturing a pharmaceutical composition for the treatment or prevention of inflammatory processes, and especially for the treatment of a DISORDER (as defined above);
(ii) a method for the treatment or prevention of inflammatory processes, especially for the treatment of a DISORDER (as defined above), which method includes administration of a pharmaceutically effective amount of a compound of the general formula (I), where the amount alleviates or suppresses the disease or the symptoms, and where the compound is given to a patient, preferably a mammal, in particular a human, requiring such a treatment;
(iii) a pharmaceutical composition having an antiinflammatory effect, in particular for the treatment of a DISORDER (as defined above), where the composition includes one of the compounds of the invention of the general formula (I) or mixtures thereof and, where appropriate, at least one pharmaceutical excipient and/or carrier.

Satisfactory results are generally to be expected in animals when the daily doses include a range from 1 μg to 100 000 μg of the compound of the invention per kg of body weight. For larger mammals, for example humans, a recommended daily dose is in the range from 1 μg to 100 000 μg per kg of body weight. A dose of 10 to 30 000 μg per kg of body weight is preferred, and a dose of 10 to 10 000 μg per kg of body weight is more preferred. This dose is for example expediently administered more than once a day. For the treatment of acute shock (e.g. anaphylactic shock) it is possible to give single doses which are distinctly higher than the abovementioned doses.

The pharmaceutical products based on the novel compounds are formulated in a manner known per se by processing the active ingredient with the carrier substances, fillers, substances influencing disintegration, binders, humectants, lubricants, absorbents, diluents, masking flavors, colorants etc. which are in use in pharmaceutical technology, and converting into the desired administration form. Reference should be made in this connection to Remington's Pharmaceutical Science, 15th ed. Mack Publishing Company, East Pennsylvania (1980).

Particularly suitable for oral administration are tablets, coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injections or suspensions and corresponding depot preparations can be used for intramuscular injection.

The novel compounds can be used for rectal administration in the form of suppositories, capsules, solutions (e.g. in the form of enemas) and ointments both for systemic and for local therapy.

The novel compounds can be used in the form of aerosols and inhalations for pulmonary administration thereof.

For local use on eyes, the external auditory canal, middle ear, nasal cavity and paranasal sinuses, the novel compounds can be used as drops, ointments, tinctures and gels in appropriate pharmaceutical preparations.

Formulations possible for topical application are gels, ointments, greasy ointments, creams, pastes, dusting powders, suspensions, emulsions and solutions. The dosage of the compounds of the general formula (I) in these preparations should be 0.01%-20% in order to achieve an adequate pharmacological effect.

The invention likewise includes the compounds of the invention of the general formula (I) as therapeutic active ingredient. The invention further includes the compounds of the invention of the general formula (I) as therapeutic active ingredient together with one or more pharmaceutically suitable and acceptable excipients and/or carriers.

The compounds of the invention of the general formula (I) can also where appropriate be formulated and/or administered in combination with further active ingredients.

The invention therefore also relates to combination therapies or combined compositions in which a compounds of the general formula (I) or a pharmaceutically acceptable salt thereof, or a pharmaceutical composition comprising a compound of the general formula (I) or a pharmaceutically acceptable salt thereof, is administered either simultaneously (where appropriate in the same composition) or successively together with one or more medicaments for the treatment of one of the pathological states mentioned above. For the treatment of rheumatoid arthritis, osteoarthritis, COPD (chronic obstructive pulmonary disorder), asthma or allergic rhinitis, for example, it is possible to combine a compound of the general formula (I) of the present invention with one or more medicaments for the treatment of such a condition. Where such a combination is administered by inhalation, the medicament to be combined can be selected from the following list:
a PDE4 inhibitor including an inhibitor of the PDE4D isoform;
a selective β.sub2.adrenoceptor agonist such as, for example, metaproterenol, isoproterenol, isoprenaline, albuterol, salbutamol, formoterol, salmeterol, terbutaline, orciprenaline, bitolterol mesylate, pirbuterol or indacaterol;
a muscarine receptor antagonist (for example an M1, M2 or M3 antagonist, such as, for example, a selective M3 antagonist) such as, for example, ipratropium bromide, tiotropium bromide, oxitropium bromide, pirenzepine or telenzepine;
a modulator of chemokine receptor function (such as, for example, a CCR1 receptor antagonist); or
an inhibitor of p38 kinase function.

For another aspect of the present invention, such a combination with a compound of the general formula (I) or a pharmaceutically acceptable salt thereof is employed for the treatment of COPD, asthma or allergic rhinitis and can be administered by inhalation or orally in combination with xanthine (such as, for example, aminophylline or theophylline), which can likewise be administered by inhalation or orally.

### EXAMPLES

The cis/trans nomenclature used in the examples below refers to the position of the substituents in position 1 and 2 of the saturated ring of the tetrahydronaphthalene system. In this connection, cis means that the highest priority substituent (according to the Cahn-Ingold-Prelog definition) on carbon atom 1 is located in the axial position and the highest priority substituent on carbon atom 2 is located in the equatorial position; or that the highest priority substituent (according to the Cahn-Ingold-Prelog definition) on carbon atom 1 is located in the equatorial position and the highest priority substituent on carbon atom 2 is located in the axial position. Correspondingly, trans means that the two highest priority substituents in each case on carbon atom 1 and carbon atom 2 are located either both in the axial position or both in the equatorial position.

### Example 1

Synthesis of Compounds of the General Formula (III)

### 6-Fluoro-5-methoxy-4,4-dimethyl-2-trifluoromethyl-1,2,3,4-tetrahydronaphthalene-1,2-diol (cis and trans isomers)

1.75 g (5.67 mmol) of 4-(3-fluoro-2-methoxyphenyl)-2-hydroxy-4,4-dimethyl-2-trifluoromethylpentanal are dissolved in 20 ml of dichlormethane, and 2.6 ml of trifluoroacetic acid are added. The reaction is stirred at room temperature under a nitrogen atmosphere for 24 h. For workup, the reaction solution is evaporated with toluene in vacuo three times and then purified by chromatography: 1.28 g of cis-6-fluoro-5-methoxy-4,4-dimethyl-2-trifluoromethyl-1,2,3,4-tetrahydro-naphthalene-1,2-diol and 300 mg of trans-6-fluoro-5-methoxy-4,4-dimethyl-2-trifluoromethyl-1,2,3,4- tetrahydronaphthalene-1,2-diol (90%). cis-6-Fluoro-5-methoxy-4,4-dimethyl-2-trifluoromethyl-1,2,3,4-tetrahydronaphthalene-1,2-diol: ¹H-NMR (300 MHz, CDCl₃): δ/ppm=1.45 (s, 3H), 1,59 (5, 3H), 1.81(d, 1H), 2.08 (d, 1H), 2.56 (d, 1H), 3.18 (d, 1H), 3.94 (d, 3H), 5.03 (d, 1H), 7.03 (dd, 1H), 7.31 (ddd, 1H). trans-6-Fluoro-5-methoxy-4, 4-dimethyl-2-trifluoro-methyl-1,2,3,4-tetrahydronaphthalene-1,2-diol: ¹H-NMR (300 MHz, CDCl₃): δ/ppm=1.52 (s, 3H), 1.55 (s, 3H), 1.83 (dd, 1H), 1.87-1.90 (m, 2H), 2.42 (d, 1H), 3.95 (d, 3H), 4.68 (dd, 1H), 6.99-7.06 (m, 2H).

The following compounds are obtained in analogy to the above method:

6-Fluoro-5-methoxy-4,4-dimethyl-2-trifluoromethyl-1,2,3,4-tetrahydronaphthalene-1,2-diol (cis and trans isomer mixture)

7-Methoxy-4,4-dimethyl-2-trifluoromethyl-1,2,3,4-tetrahydronaphthalene-1,2-diol (cis and trans isomer mixture)

### Example 2

Synthesis of Compounds of the General Formula (IV)

### Example 2A

### cis-1-Amino-6-fluoro-5-methoxy-4,4-di-methyl-2-trifluoromethyl-1,2,3,4-tetrahydro-naphthalen-2-ol

a) Benzyl 7-fluoro-6-methoxy-5,5-dimethyl-2,2-dioxo-3a-trifluoromethyl-3a,4,5,9b-tetrahydro-3-oxy-2lambda*6*-thia-1-aza-cyclopenta[a]naphthalene-1-carboxylate 2.00 g (6.48 mmol) of 6-fluoro-5-methoxy-4,4-dimethy1- 2-trifluoromethyl-1,2,3,4-tetrahydronaphthalene-1,2- diol and 5.12 g (16.2 mmol) of Burgess's reagent are dissolved in 50 ml of THF and stirred under a nitrogen atmosphere at 65-70° C. for 7 h and then at room temperature for 12 h. The reaction solution is evaporated in vacuo, and the residue is purified by chromatography: 1.66 g (51%). ¹H-NMR (300 MHz, CDCl₃): δ/ppm=1.54 (s, 3H), 1.59 (s, 3H), 2.01 (d, 1H), 2.29 (d, 1H), 3.95 (d, 3H), 5.36 (d, 1H), 5.44 (d, 1H), 5.95 (a, 1H), 6.97 (dd, 1H), 7.19 (dd, 1H), 7.35-7.42 (m, 5H).

b) cis-1-Amino-6-fluoro-5-methoxy-4,4-dimethyl-2-tri-fluoromethyl-1,2,3,4-tetrahydronaphthalen-2-ol 1.2 g (2.38 mmol) of benzyl 7-fluoro-6-methoxy-5,5-dimethyl-2,2-dioxo-3a-trifluoromethyl-3a,4,5,9b-tetrahydro-3-oxy-2lambda*6*-thia-1-aza-cyclopenta[alnaphthalene-1-carboxylate are dissolved in 12 ml of dioxane and, after addition of 8 ml of 4N HCl solution, treated in a microwave at 250 watt and 140° C. for 20 minutes twice. For workup, the solution is evaporated in vacuo, and the residue is adjusted to pH 14 with 4N sodium hydroxide solution at 0° C. and extracted three times with ethyl acetate. The combined organic phases are washed with saturated sodium chloride solution, dried over sodium sulfate, filtered and concentrated in vacuo: 41% of the desired product. ³H-NMR (300 MHz, CDCl₃): δ/ppm=1.36 (s, 3H), 1.49 (s, 3H), 1.78 (s, 1H), 1.95 (d, 1H), 3.83 (d, 3H), 4.03 (s, 1H), 7.12 (dd, 1H), 7.51 (dd, 1H).

### Example 2B

### trans-1-Amino-6-fluoro-5-methoxy-4,4-dimethyl-2-trifluoromethyl-1,2,3,4-tetrahydro- naphthalen-2-ol

a) 1-Azido-6-fluoro-5-methoxy-4,4-dimethyl-2-trifluoro-methyl-1,2,3,4-tetrahydronaphthalen-2-ol 420 mg (1.27 mmol) of tetrabromomethane and 454 mg (1.14 mmol) of 1,2-bis(diphenylphoshino)ethane (DiPhos) are added to a solution of 130 mg (0.422 mmol) of 6-fluoro-5-methoxy-4,4-dimethyl-2-trifluoromethyl-1,2,3,4-tetrahydronaphthalene-1,2-cis-diol in 4.5 ml of dichlormethane at room temperature under a nitrogen atmosphere. It is possible analogously to start this reaction also with the corresponding 1,2-cis/trans-diol mixture instead of the 1,2-cis-diol. After stirring at room temperature for 3 h, 20 ml of ether are added and, after stirring for 5 min, the resulting precipitate is filtered off and washed with ether, and the solvent is evaporated in vacuo: 200 mg of crude product which is employed without further purification in the next reaction.

190 mg (0.512 mmol) of crude product are mixed with 2 ml of a sodium azide-containing DMSO solution (150 mg of sodium azide in 5 ml of dimethyl sulfoxide are stirred at room temperature for 24 h). The reaction solution is stirred at room temperature for 3 h and at 40-45° C. for 3 h under a nitrogen atmosphere. For workup, 5 ml of water are added to the reaction mixture and it is extracted three times with ethyl acetate. The combined organic phases are washed with saturated sodium chloride solution, dried over sodium sulfate, filtered, concentrated in vacuo and purified by column chromatography: 80 mg of the title compound. ¹H-NMR (300 MHz, CDCl₃): δ/ppm=1.52 (B, 3H), 1.56 (a, 3H), 1.84 (dd, 1H), 1.97 (s, 1H), 2.25 (d, 1H), 3.98 (d, 3H), 4.48 (d, 1H), 6.93 (dd, 1H), 7.07 (dd, 3H).

b) trans-i-Amino-6-fluoro-5-methoxy-4,4-dimethyl- 2-trifluoromethyl-1,2,3,4-tetrahydronaphthalen-2-ol 311 mg (4.76 mmol) of zinc powder are added to a solution of 1.22 g (3.66 mmol) of 1-azido-6-fluoro-5-methoxy-4,4-dimethyl-2-trifluoromethyl-1,2,3,4-tetrahydronaphthalen-2-ol and 450 mg (8.42 mmol) of ammonium chloride in 9.7 ml of ethanol and 3.3 ml of water, and the mixture is then stirred at 90° C. under a nitrogen atmosphere for 30 min. After cooling to room temperature, 25 ml of ethyl acetate and 1.22 ml of ammonia solution are added, and the mixture is stirred for 5 min and filtered. The filtrate is washed with saturated sodium chloride solution, dried over sodium sulfate, filtered and concentrated in vacuo. The crude product is purified by column chromatography: 950 mg of the title compound. ¹H-NMR (300 MHz, CDCl₃): δ/ppm=1.42 (a, 3H), 1.46 (s, 3H), 1.61 (dd, NH), 1.77 (bs, 2H), 2.20 (d, 1H), 3.84 (d, 3H), 3.88 (s, 1H), 5.63 (s, 1H), 6.98 (dd, 1H), 7.10 (dd, 1H).

### Example 3

Synthesis of Compounds of the General Formula (I)

### Example 3A

### N-(cis-6-fluoro-2-hydroxy-5-methoxy-4,4-dimethyl-2-trifluoromethyl-1,2,3,4-tetrahydro-naphthalen-1-yl)naphthalene-2-carboxamide

A solution of 4-dimethylaminopyridine (73 mg, 0.60 mmol) in DMF (0.4 ml) and a solution of 2-naphthalenecarbonyl chloride (84 mg, 0.44 mmol) in DMF (0.8 ml) are successively added to a solution of cis-1-amino-6-fluoro-5-methoxy-4,4-dimethyl-2-trifluoromethyl-1,2,3,4-tetrahydronaphthalen-2-ol (100 mg, 0.40 mmol) in DMF (0.8 ml), and the resulting mixture is stirred at room temperature overnight. For workup, sodium bicarbonate solution (3 ml, half-saturated solution in water) and ethyl acetate (6 ml) are added and, after extraction, the organic phase is isolated and concentrated. One third of the resulting crude product is purified by HPLC-MS and affords 28 mg of the title compound. 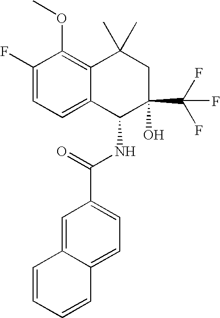
HPLC: 3.4 min (method A).

MS (ESI): m/z 461.

¹H-NMR (CDCl₃, 400 MHz): δ/ppm=1.52 (s, 3H), 1.67 (s, 3H), 2.12 (m, 2H), 3.30 (broad s), 3.97 (s br, 3H), 5.67 (d, 1H), 6.93-7.07 (m, 3w), 7.57 (m, 2H), 7.91 (m, 4H), 8.36 (s, 1H).

### Example 3B

### N-(cis-6-fluoro-2,5-dihydroxy-4,4-dimethyl-2-trifluoromethyl-1,2,3,4-tetrahydronaphthalen-1-yl)naphthalene-2-carboxamide

The remaining two thirds of the crude product from the synthesis described above in example 3A are dissolved in dichloromethane (0.5 ml) and cooled to −40° C. and, at this temperature, boron tribromide solution (1 ml, 1.0M in dichloromethane, 1.0 mmol) is added, and the mixture is warmed to room temperature while stirring overnight.

For workup, potassium carbonate solution (1.5 ml, half-saturated solution in water) is added in an ice bath, the resulting mixture is stirred in the ice bath for 15 min and diluted and extracted with ethyl acetate (3 ml), and the organic phase is isolated and concentrated. The residue is purified by HPLC-MS and affords 88 mg of the title compound. 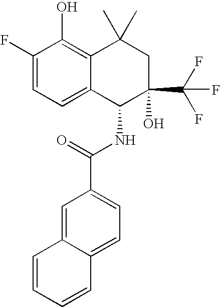
HPLC: 3.1 min (method A).

MS (ESI): m/z 447.

¹H-NMR (CDCl₃, 400 MHZ): δ/ppm 1.5G (B, 3H), 1.69 (s, 3H), 2.13 (s, 2H), 5.43 (d, 1H), 5.66 (d, 1H), 6.88-7.03 (m, 3H), 7.57 (m, 2H) , 7.92 (m, 4H) , 8.37 (s, 1H)

The following compounds of the general formula (I) were obtained in analogy to examples 3A and 3B described above:

| Ex. | Structure | Name | MS (ESI) | HPLC |
|---|---|---|---|---|
| 3C | 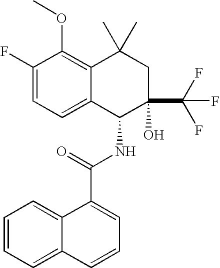 | N-(cis-6-fluoro-2- hydroxy-5-methoxy-4,4- dimethyl-2-trifluoro- methyl-1,2,3,4- tetrahydronaphthalen- 1-yl)naphthalene-1- carboxamide | 461 | 3.3 min. (method A) |
| 3D | 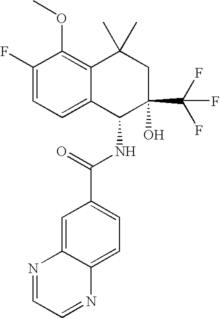 | N-(cis-6-fluoro-2- hydroxy-5-methoxy-4,4- dimethyl-2-trifluoro- methyl-1,2,3,4- tetrahydronaphthalen- 1-yl)quinoxaline-6- carboxamide | 463 | 2.9 min. (method A) |
| 3E | 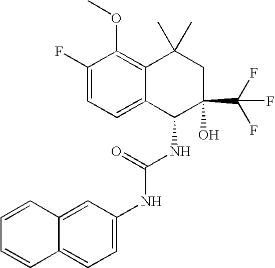 | 1-cis-6-fluoro-2- hydroxy-5-methoxy-4,4- dimethyl-2-trifluoro- methyl-1,2,3,4- tetrahydronaphthalen- 1-yl)-3-naphthalen-2- ylurea | 476 | 3.4 min. (method A) |
| 3F | 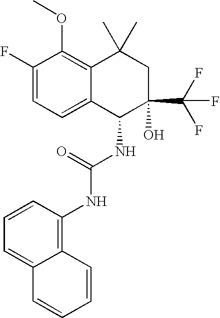 | 1-(cis-6-fluoro-2- hydroxy-5-methoxy-4,4- dimethyl-2-trifluoro- methyl-1,2,3,4- tetrahydronaphthalen- 1-yl)-3-naphthalen-1- ylurea | 476 | 3.3 min. (method A) |
| 3G | 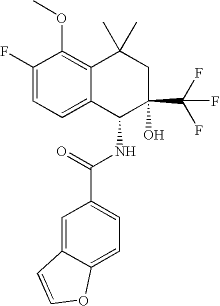 | N-(cis-6-fluoro-2- hydroxy-5-methoxy-4,4- dimethyl-2-trifluoro- methyl-1,2,3,4- tetrahydronaphthalen- 1-yl)benzofuran-5- carboxamide | 451 | 3.2 min. (method A) |
| 3H | 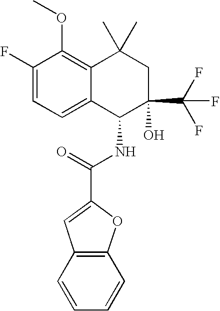 | N-(cis-6-fluoro-2- hydroxy-5-methoxy-4,4- dimethyl-2-trifluoro- methyl-1,2,3,4- tetrahydronaphthalen- 1-yl)benzofuran-2- carboxamide | 451 | 3.3 min. (method A) |
| 3I | 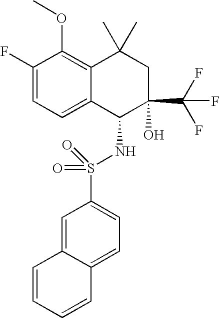 | N-(cis-6-fluoro-2- hydroxy-5-methoxy-4,4- dimethyl-2-trifluoro- methyl-1,2,3 4- tetrahydronaphthalen- 1-yl)naphthalene-2- sulfonamide | 498 | 3.5 min. (method A) |
| 3J | 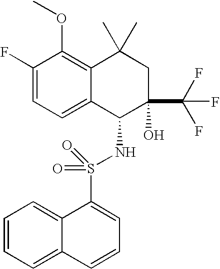 | N-(cis-6-fluoro-2- hydroxy-5-methoxy-4,4- dimethyl-2-trifluoro- methyl-1,2,3,4- tetrahydronaphthalen- 1-yl)naphthalene-1- sulfonamide | 498 | 3.5 min. (method A) |
| 3K | 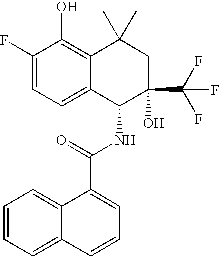 | N-(cis-6-fluoro-2,5- dihydroxy-4,4- dimethyl-2-trifluoro- methyl-1,2,3,4- tetrahydronaphthalen- 1-yl)naphthalene-1- carboxamide | 448 | 11.2 min. (method C) |
| 3L | 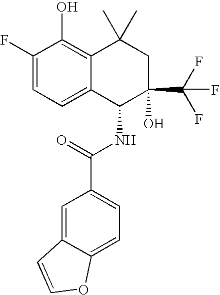 | N-(cis-6-fluoro-2,5- dihydroxy-4,4- dimethyl-2-trifluoro- methyl-1,2,3,4- tetrahydronaphthalen- 1-yl)benzofuran-5- carboxamide | 438 | 10.3 min. (method C) |
| 3M | 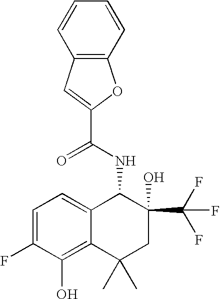 | N-(cis-6-fluoro-2,5- dihydroxy-4,4- dimethyl-2-trifluoro- methyl-1,2,3,4- tetrahydronaphthalen- 1-yl)benzofuran-2- carboxamide | 438 | 10.8 min. (method C) |
| 3N | 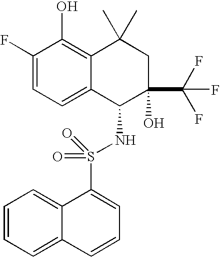 | N-(cis-6-fluoro-2,5- dihydroxy-4,4- dimethyl-2-trifluoro- methyl-1,2,3,4- tetrahydronaphthalen- 1-yl)naphthalene-1- sulfonamide | 483 | 3.2 min. (method A) |
| 3O | 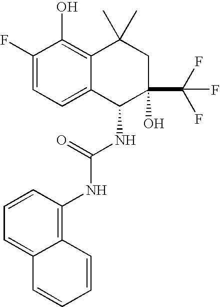 | 1-(cis-6-fluoro-2,5- dihydroxy-4,4- dimethyl-2- trifluoromethyl- 1,2,3,4-tetrahydro- naphthalen-1-yl)-3- naphthalen-1-ylurea | 463 | 10.7 min. (method C) |
| 3P | 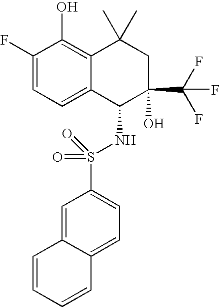 | N-(cis-6-fluoro-2,5- dihydroxy-4,4- dimethyl-2-trifluoro- methyl-1,2,3,4- tetrahydronaphthalen- yl)naphthalene-2- sulfonamide | 483 | 3.2 min. (method A) |
| 3Q | 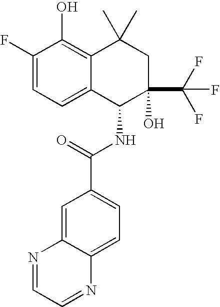 | N-(cis-6-fluoro-2,5- dihydroxy-4,4- dimethyl-2-trifluoro- methyl-1,2,3,4- tetrahydronaphthalen- 1-yl)quinoxaline-6- carboxamide | 450 | 8.3 min. (method C) |
| 3R | 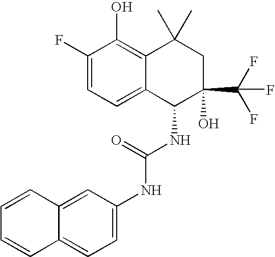 | 1-(cis-6-fluoro-2,5- dihydroxy-4,4- dimethyl-2- trifluoromethyl- 1,2,3,4-tetrahydro- naphthalen-1-yl)-3- naphthalen-2-ylurea | 463 | 9.5 min. (method E) |
| 3S | 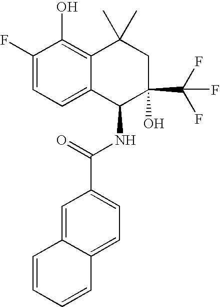 | N-(trans-6-fluoro-2,5- dihydroxy-4,4- dimethyl-2-trifluoro- methyl-1,2,3,4- tetrahydronaphthalen- 1-yl)naphthalene-2- carboxamide | 447 | 2.9 min. (method A) |
| 3T | 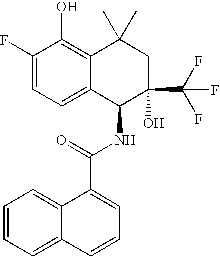 | N-(trans-6-fluoro-2,5- dihydroxy-4,4- dimethyl-2-trifluoro- methyl-1,2,3,4- tetrahydronaphthalen- 1-yl)naphthalene-1- carboxamide | 448 | 8.8 min. (method B) |
| 3U | 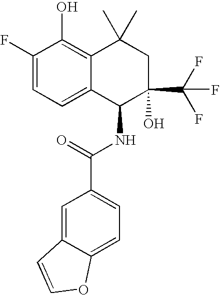 | N-(trans-6-fluoro-2,5- dihydroxy-4,4- dimethyl-2-trifluoro- methyl-1,2,3,4- tetrahydronaphthalen- 1-yl)benzofuran-5- carboxamide | 437 | 2.8 min. (method A) |
| 3V | 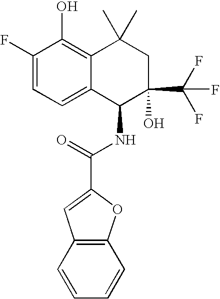 | N-(trans-6-fluoro-2,5- dihydroxy-4,4- dimethyl-2-trifluoro- methyl-1,2,3,4- tetrahydronaphthalen- 1-yl)benzofuran-2- carboxamide | 438 | 3.0 min. (method A) |
| 3W | 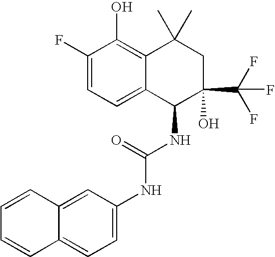 | 1-(trans-6-fluoro-2,5- dihydroxy-4,4- dimethyl-2- trifluoromethyl- 1,2,3,4-tetrahydro- naphthalen-1-yl)-3- naphthalen-2-ylurea | 463 | 9.2 min. (method B) |
| 3X | 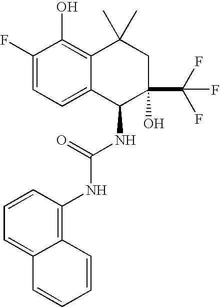 | 1-(trans-6-fluoro-2,5- dihydroxy-4,4- dimethyl-2- trifluoromethyl- 1,2,3,4-tetrahydro- naphthalen-1-yl)-3- naphthalen-1-ylurea | 463 | 8.9 min. (method B) |
| 3Y | 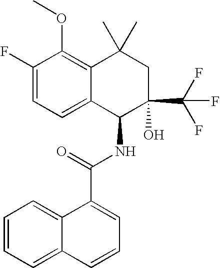 | N-(trans-6-fluoro-2- hydroxy-5-methoxy-4,4- dimethyl-2-trifluoro- methyl-1,2,3,4- tetrahydronaphthalen- 1-yl)naphthalene-1- carboxamide | 462 | 2.8 min. (method D) |
| 3Z | 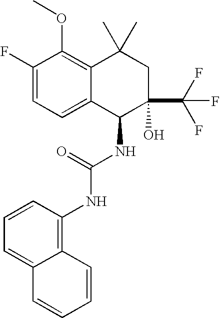 | 1-(trans-6-fluoro-2- hydroxy-5-methoxy-4,4- dimethyl-2- trifluoromethyl- 1,2,3,4-tetrahydro- naphthalen-1-yl)-3- naphthalen-1-ylurea | 476 | 3.1 min. (method A) |

### Example 4

Synthesis of Further Compounds of the General Formula (I)

### Example 4

### 6-Fluoro-5-methoxy-4,4-dimethyl-1-[(pyrazolo[1,5-a]pyridin-3ylmethyl)amino]-2-trifluoromethyl-1,2,3,4-tetrahydronaphthalen-2-ol, (cis isomer)

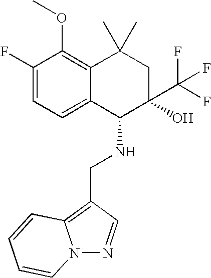 cis-1-Amino-6-fluoro-5-methoxy-4,4-dimethyl-2-trifluoromethyl-1,2,3,4- tetrahydronaphthalen-2-ol (62 mg, 0.20 mmol) and pyrazolo[1,5-a]pyridine-3-carbaldehyde (30 mg (0.20 mmol) are dissolved in xylene (5.0 ml) and, after addition of titanium tetraethoxide (0.085 ml, 0.40 mmol), stirred at 150° C. for 3 hours. After removal of the solvent, the residue is taken up in methanol (1.0 ml) and tetrahydrofuran (1.0 ml), and sodium borohydride (22 mg, 0.58 mmol) is added. The reaction is stopped after 3 hours at room temperature by adding water. Methanol and tetrahydrofuran are removed in vacuo. The aqueous phase is extracted with dichloromethane, and the organic phase is dried over sodium sulfate. Removal of the solvent and subsequent purification by chromatography (silica gel, hexane/ethyl acetate 3:7) result in 26 mg of the title compound.

¹H-NMR (300 MHz, d₆-DMSO): δ/ppm=1.39 (s, 3H), 1.46 (s, 3H), 1.85 (d, 1H), 2.07 (d, 1H), 3.83 (d, 3H), 3.94 (s, 1H), 3.98 (d, 1H), 4.06 (d, l), 5.92 (br, 1H), 6.85 (td, 1H), 7.07-7.23 (m, 3H), 7.63 (d, 1H), 7.98 (S, 1H), 8.62 (d, 1H).

Description of the HPLC Methods

Method A:

Waters Alliance 2795, Waters Photo Diode Array 2996 (200-320 nm), Micromass ZQ; column Micra NPS ODS 2 (33×4.6 mm, 1.5 μm); gradient 0-90% acetonitrile (0.01t formic acid) in water (0.01% formic acid) (4.5 min.), 90% acetonitrile (0.01% formic acid) in water (0.01% formic acid) (2 min); flow rate 0.8 ml/min.

Method B:

Waters Pump 515, Waters Dual Absorbance Detector 2487 (254 nm), Micromass ZQ; column X-Terra (150×4.6 mm, 5 μm); gradient 54-95% acetonitrile (0.01% formic acid) in water (0.01% formic acid) (10 min); flow rate 1 ml/min.

Method C:

Waters Pump 616, Hitachi L-4000 (254 nm); column Chromasil C8 (150×4.6 mm, 5 μm); gradient 30-95% acetonitrile (0.01% formic acid) in water (0.01% formic acid) (15 min.), 95% acetonitrile (0.01% formic acid) in water (0.01% formic acid) (15 min); flow rate 1 ml/min.

Method D:

Hewlett-Packard 1100 Pump, HP1100 Detector (200-320 nm), Micromass PLCZ; column Micra NPS ODS 2 (33×4.6 mm, 1.5 μm); gradient 0-90% acetonitrile (0.01% trifluoroacetic acid) in water (0.01% trifluoroacetic acid) (4.5 min), 90% acetonitrile (0.01% trifluoroacetic acid) in water (0.01% trifluoroacetic acid) (2 min); flow rate 0.8 ml/min.

1. A compound of the general formula (I) 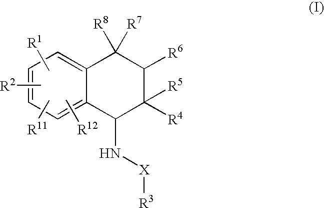
in which
R¹ and R² are independently of one another a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group,
or R¹ and R² together form a group selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)—(CH₂)ₙ₊₁—and —NH—N═CH—, where n is 1 or 2, and the terminal oxygen atoms and/or carbon atoms and/or nitrogen atoms are linked to directly adjacent ring carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a C₁-C₁₀-alkyl group which is optionally substituted by 1 to 3 hydroxy groups, 1 to 3 halogen atoms, and/or 1 to 3 (C₁-C₅)-alkoxy groups, an optionally substituted (C₃-C₇)-cycloalkyl group, an optionally substituted heterocyclyl group, an optionally substituted aryl group, or a mono- or bicyclic heteroaryl group which is optionally substituted by one or more groups which are selected independently of one another from
(C₁-C₅)-alkyl groups which themselves may optionally be substituted by 1 to 3 hydroxy or 1 to 3 —COOR¹³ groups,
(C₁-C₅)-alkoxy groups, halogen atoms, hydroxy groups, —NR⁹R^{9a} groups,
and
exo methylene groups and optionally comprises 1 to 4 nitrogen atoms and/or 1 to 2 oxygen atoms and/or 1 to 2 sulfur atoms and/or 1 to 2 keto groups, this group being linked by any position to the group X and possibly being optionally hydrogenated at one or more positions,
R⁴ is a hydroxy group, an —OR¹⁰ group or an —O(CO)R¹⁰ group,
R⁵ is a (C₁-C₁₀ )-alkyl group which is optionally partly or completely fluorinated, a (C₃-C₇) cycloalkyl group, a (C₁-C₈)alkyl-(C₃-C₇) cycloalkyl group, a (C₂-C₈)alkenyl-(C₃-C₇) cycloalkyl group, a heterocyclyl group, a (C₁-C₈)alkylheterocyclyl group, a (C₂-C₈)-alkenylheterocyclyl group, an aryl group, a (C₁-C₈)alkylaryl group, a (C₂-C₈)alkenylaryl group, a (C₂-C₈)-alkynylaryl group, a mono- or bicyclic heteroaryl group which is optionally substituted by 1 to 2 keto groups, 1 to 2 (C₁-C₅)-alkyl groups, 1 to 2 (C₁-C₅)-alkoxy groups, 1 to 3 halogen atoms, and/or 1 to 2 exo methylene groups and comprises 1 to 3 nitrogen atoms and/or 1 to 2 oxygen atoms and/or 1 to 2 sulfur atoms, a (C₁-C₈)alkylheteroaryl group, a (C₂-C₈)alkenylheteroaryl group, or a (C₂-C₈) alkynylheteroaryl group, this group being linked by any position to the tetrahydronaphthalene system and possibly being optionally hydrogenated at one or more positions,
R⁶ is a hydrogen atom, a halogen atom, or an optionally substituted (C₁-C₁₀)alkyl group,
R⁷ and R⁸ are independently of one another a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (C₃-C₆)-cyclalkyl ring; or
R⁶ and R⁷ together form a fused five- to eight-membered saturated or unsaturated carbocycle or heterocycle is which is optionally substituted by 1 to 2 keto groups, 1 to 2 (C₁-C₈)-alkyl groups, 1 to 2 (C₁-C₅)-alkoxy groups, and/or 1 to 4 halogen atoms; or
R¹ and R⁸ together form a fused five- to eight-membered saturated or unsaturated carbocycle or heterocycle which is optionally substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a} are independently of one another a hydrogen atom, (C₁-C₅)-alkyl or —(CO)—(C₁-C₅)-alkyl,
R¹⁰ is a (C₁-C₁₀)-alkyl group or any hydroxy protective group,
R¹³ is a hydrogen atom or a (C₁-C₅)-alkyl group, and
x is a group —C(—O)—, —C(═S)—, —C(═o)—NH—, —C(—S)—NH—, —S(O)ₘ (where m=1 or 2), —C(═O)—O—, —C(═S)—O— or a group —(CH₂)ₚ— (where p=1, 2 or 3), where if X comprises a carbonyl or thiocarbonyl function, this function is linked to the group —NH— in the general formula (I),
with the proviso that if R³ is an optionally substituted (C₁-C₁₀)-alkyl group, X cannot be a —(CH₂)ₚ— group; in the form of any stereoisomer or of a mixture of stereoisomers; or as pharmacologically acceptable salt or derivative.

2. A compound as claimed in claim 1, where
X is a group —C(═O)—, —C(═O)—NH—, —SO₂— or —CH₂—.

3. A compound as claimed in claim 1 or 2, where
R⁴ is a hydroxy group or a group —OR¹⁰.

4. A compound as claimed in any of the preceding claims, where
R⁵ is a (C₁-C₁₀)-alkyl group which is optionally partly or completely fluorinated.

5. A compound as claimed in any of the preceding claims, where
R⁷ and R⁸ are each a methyl group, or together with the carbon atom of the tetrahydronaphthalene system form a cyclopropyl group.

6. A compound as claimed in any of the preceding claims, where
R³ is an optionally substituted aryl or heteroaryl group.

7. A compound as claimed in claim 6, where the optionally substituted aryl or heteroaryl group is selected from the group consisting of naphthyl, benzofuranyl, pyrazolo[1,5-a]pyridinyl, phenyl, phthalidyl, isoindolyl, dihydroindolyl, dihydro-isoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, guinoxalinyl, cinnolinyl, phthalazinyl, 1,7- or 1,8-naphthyridinyl, dihydroindolonyl, dihydroisoindolonyl, chromanyl, isochromanyl, benzimidazole or indolyl group.

8. A compound as claimed in any of the preceding claims for manufacturing a medicament.

9. The use of a compound as claimed in any of claims 1 to 7 for manufacturing a pharmaceutical composition for the treatment or prevention/ of inflammatory processes.

10. A method for the treatment or prevention of inflammatory processes in a patient, characterized in that a pharmaceutically effective amount of a compound of the general formula (I) as claimed in any of claims 1 to 7 is administered to a patient requiring such a treatment or prevention.

11. A pharmaceutical product comprising at least one compound as claimed in any of claims 1 to 7 and one or more pharmaceutically acceptable carriers and/or excipients.

12. A process for preparing compounds as claimed in any of claims 1 to 7, characterized in that
a) an open-chain carbonyl compound of the general formula (II) 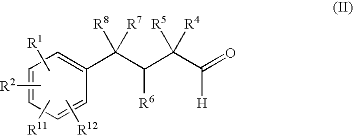
is cyclized where appropriate with addition of an inorganic or organic acid or Lewis acid to a compound of the general formula (III) 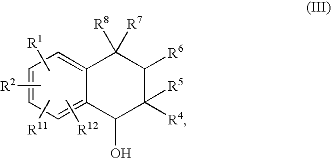
b) the compound of the general formula (III) is converted by replacing the hydroxy group by the amino group into a compound of the general formula (IV) 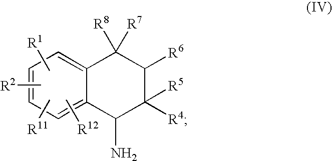
and
c) the compound of the general formula (IV) is converted by reaction with a compound is selected from compounds of the general formulae R³-X-Nu (where Nu is a nucleofugic group), R³—N═C═O or R³—N═C═S, and where appropriate subsequent replacement of a carbonyl oxygen atom by sulfur, into a compound of the general formula (I),
where the substituents R¹ to R¹² and X have the meanings indicated in claims 1 to 7.

13. A process for preparing compounds as claimed in any of claims 1 to 7, characterized in that a compound of the general formula (IV) as described in claim 12 is reacted with a compound of the general formula R³—(CH₂)ᵥ—CHO (where v is 0, 1 or 2), and the resulting imine is hydrogenated to give a compound of the general formula (I), where X has the meaning —(CH₂)ₚ-indicated in claim 1, and the substituents R³ to R¹² have the meanings indicated in claims 1 to 7.

14. A process for preparing compounds according to any of claims 1 to 7, characterized in that a compound of the general formula (IV) as described in claim 12 is reacted with phosgene or thiophosgene, and the resulting isocyanate or isothiocyanate is subsequently reacted with compounds of the general formula R³—OH or R³—NH₂ to give a compound of the general formula (I), where X has the meaning —C(═O)—NH—, —C(═S)—NH— or —C(═O)—O— indicated in claim 1, and the substituents R¹ to R¹² have the meanings indicated in claims 1 to 7.

15. A process for preparing a compound as claimed in any of claims 1 to 7, characterized in that
a) a compound of the general formula (VI) 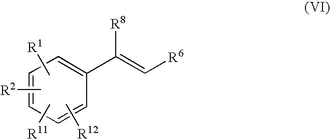
where R⁸ is a (C₁-C₁₀)-alkyl group, is reacted with an α-keto carboxylic acid or an α-keto carboxylic ester of the general formula R⁵—C(═O)—COOR¹³ in an ene reaction in the presence of an optionally chiral Lewis acid to give a compound of the general formula (VII) 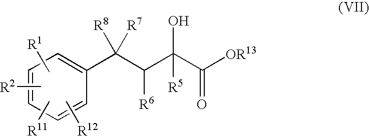
in which R⁷ and R⁸ together have the meaning of a (C₁-C₁₀)-alkylidene group,
b) the compound of the general formula (VII) is reduced to an aldehyde of the general formula (IIa), 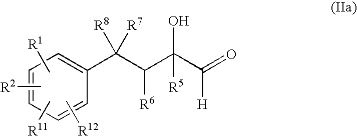
in which R⁷ and R⁸ together have the meaning of a (C₁-C₁₀)-alkylidene group., and
c) the aldehyde (IIa) is converted in analogy to the process indicated in claim 12 into a compound of the general formula (I) in which R⁷ and R⁸ together have the meaning of a (C₁-C₁₀)-alkylidene group,
where the substituents R¹ to R¹³ have the meanings indicated in claims 1 to 7.

16. The use of compounds of the general formula (IV) 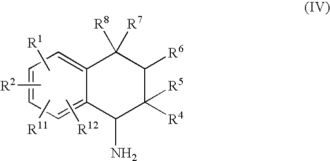
where the substituents R¹ to R¹² have the meanings indicated in claims 1 to 7, for preparing compounds of the general formula (I) as claimed in any of claims 1 to 7.

17. A compound of the formula (IV), 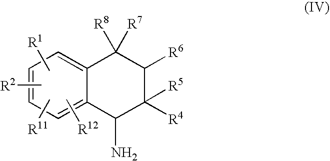
where the substituents R¹ to R¹² have the meanings indicated in claims 1 to 7.
